(19) United States
(12) Patent Application Publication
(10) Pub. No.: US 2004/0009222 A1
(43) Pub. Date: Jan. 15, 2004

(54) Processes for forming a drug delivery device

(75) Inventors: Kang-Jye Chou, Watertown, MA (US); Hong Guo, Belmont, MA (US); Paul Ashton, Boston, MA (US); Robert W. Shimizu, Acton, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA

(21) Appl. No.: 10/428,214

(22) Filed: May 2, 2003

Related U.S. Application Data
(60) Provisional application No. 60/377,974, filed on May 7, 2002.
(60) Provisional application No. 60/437,576, filed on Dec. 31, 2002.
(60) Provisional application No. 60/452,348, filed on Mar. 6, 2003.

(51) Int. Cl. A61K 31/7072; A61K 31/58; A61K 9/24

(52) U.S. Cl. 424/471; 514/50; 514/171

(57) ABSTRACT

A drug delivery device can, in whole or in part, be formed by co-extruding a drug core and an outer tube. The outer tube may be permeable, semi-permeable, or impermeable to the drug. The drug core may include a polymer matrix which does not significantly affect the release rate of the drug. The outer tube, the polymer matrix of the drug core, or both may be bioerodible. The co-extruded product can be segmented into drug delivery devices. The devices may be left uncoated so that their respective ends are open, or the devices may be coated with, for example, a layer that is permeable to the drug, semi-permeable to the drug, or bioerodible.

### RELATED APPLICATIONS

This application claims the benefit of U.S. Application No. 60/377,974, filed May 7, 2002; U.S. Application No. 60/437,576, filed Dec. 31, 2002; and U.S. Application No. 60/452,348, filed Mar. 6, 2003, the specifications of each of which are incorporated by reference herein.

### FIELD OF THE INVENTION

The present invention relates to processes useful for making a drug delivery device, and more particularly to processes useful for making a drug delivery device using co-extrusion for some portion of or all of such a device.

### BRIEF DESCRIPTION OF THE RELATED ART

U.S. Pat. No. 6,375,972, by Hong Guo et al., entitled SUSTAINED RELEASE DRUG DELIVERY DEVICES, METHODS OF USE, AND METHOD OF MANUFACTURING THEREOF, incorporated by reference herein in its entirety, describes certain drug delivery devices which have numerous advantages. As will be readily appreciated by those of skill in the art, however, the reduction in the size of such devices as a part of a normal product development cycle makes manufacture of the devices more difficult. As described in the '972 patent, the drug reservoir can be formed within the tube which supports it by a number of different methods, including injecting the drug matrix into the preformed tube. With smaller tubes and more viscous drug matrix materials, this step in the formation of the device becomes increasingly difficult.

A recent article by Kajihara et al. appearing in the Journal of Controlled Release, 73, pp. 279-291 (2001) describes the preparation of sustained-release formulations for protein drugs using silicones as carriers. The disclosure of this article is incorporated herein in its entirety.

There remains a need for improved techniques for preparing implantable drug delivery systems, such as devices having an inner reservoir containing at least one drug and a self-supporting tube at least partially surrounding the reservoir. There also remains a need for techniques that apply co-extrusion technology to the manufacture of such drug delivery systems.

Objects, features, and attendant advantages of the present invention will become apparent to those skilled in the art from a reading of the following detailed description of embodiments constructed in accordance therewith, taken in conjunction with the accompanying drawings.

### SUMMARY OF THE INVENTION

A drug delivery device can, in whole or in part, be formed by co-extruding a drug core and an outer tube. The outer tube may be permeable, semi-permeable, or impermeable to the drug. The drug core may include a polymer matrix which does not significantly affect the release rate of the drug. The outer tube, the polymer matrix of the drug core, or both may be bioerodible. The co-extruded product can be segmented into drug delivery devices. The devices may be left uncoated so that their respective ends are open, or the devices may be coated with, for example, a layer that is permeable to the drug, semi-permeable to the drug, or bioerodible.

Thus, in one aspect, the invention provides a method of making a drug delivery device by co-extruding an inner drug-containing core, e.g., a mixture of at least one drug and at least one polymer, and at least one outer polymeric skin that at least partially surrounds the core. The device may be insertable, injectable, or implantable. The polymer of the inner drug-containing core may be bioerodible.

In certain embodiments, the at least one drug and the at least one polymer are admixed in powder form. The drug may be a codrug or a prodrug, a steroid, such as flucinolone acetonide (FA), loteprednol etabonate, or triamcinolone acetonide (TA), or an anti-metabolite, such as 5-flurouracil (5-FU), and may be carried in the core or in the skin.

The outer polymeric skin may be impermeable, semi-permeable, or permeable to a drug disposed within the inner drug-containing core, and may comprise any biocompatible polymer, such as polycaprolactone (PCL), an ethylene/vinyl acetate copolymer (EVA), polyalkyl cyanoacralate, polyurethane, a nylon, or poly(dl-lactide-co-glycolide) (PLGA), or a copolymer of any of these. In certain embodiments, the outer polymeric skin is bioerodible. In certain embodiments, the outer polymeric skin is radiation curable and the method further comprises applying radiation to the co-extruded drug delivery device. In certain embodiments, the outer polymeric skin comprises at least one drug, such as triamcinolone acetonide (TA).

In certain embodiments, the inner drug-containing core comprises a bioerodible polymer, such as poly(vinyl acetate) (PVAC), PCL, PEG, or PLGA, and may further comprise flucinolone acetonide (FA) and/or 5-fluorouracil (5-FU).

In another aspect, the invention relates to a method of making a drug delivery device, by forwarding a polymeric material to a first extrusion device, forwarding a drug to a second extrusion device, co-extruding a mass including the polymeric material and the drug, and forming the mass into at least one co-extruded drug delivery device which comprises a core including the drug and an outer layer including the polymeric material. In certain embodiments, the drug forwarded to the second extrusion device is in admixture with at least one polymer. In certain embodiments, the drug and the at least one polymer are admixed in powder form. In certain embodiments, this act includes forwarding more than one drug to the second extrusion device. In certain embodiments, the polymeric material is one of impermeable, semi-permeable, or permeable to the drug. The polymeric material may be bioerodible and/or radiation curable. In latter instances, the method may further comprise applying radiation to the co-extruded drug delivery device.

In certain embodiments, the co-extruded drug delivery device is in a tubular form, and may be segmented into a plurality of shorter products. In certain embodiments, the method further comprises coating the plurality of shorter products with one or more layers including at least one of a layer that is permeable to the drug, a layer that is semi-permeable to the drug, and a layer that is bioerodible. The polymeric material may include any biocompatible polymer, such as polycaprolactone (PCL), an ethylene/vinyl acetate copolymer (EVA), polyalkyl cyanoacralate, polyurethane, a nylon, or poly(dl-lactide-co-glycolide) (PLGA), or a copolymer of any of these. The drug may be a steroid, such as FA or TA, or an anti-metabolite, such as 5-FU.

In certain of the above embodiments, the polymeric material includes at least one drug, such as TA and/or FA, optionally in admixture with at least one of PCL, PLGA or PVAC. In certain embodiments, the polymeric material includes at least one of PCL, PLGA or an EVA and the drug includes FA in admixture with at least one of PCL, PLGA or PVAC.

In yet another aspect, the invention provides a device for fabricating an implantable drug delivery device including a first extruder for extruding a core, wherein the core includes at least one drug, and a second extruder for extruding a skin, wherein the skin is disposed about the core to form a co-extruded material, and wherein the skin has at least one of a permeability or an erodibility selected to control the release rate of the drug in a device formed from a segment of the co-extruded material. The device may further comprise a segmenting station that separates the co-extruded material into a plurality of segments, and/or a curing station that at least partially cures the co-extruded material.

### BRIEF DESCRIPTION OF THE DRAWINGS

The invention of the present application will now be described in more detail with reference to preferred embodiments of the apparatus and method, given only by way of example, and with reference to the accompanying drawings, in which:

FIGS. 1-4 illustrate data representative of release rates for devices according to the present invention; and

FIG. 5 schematically illustrates an exemplary apparatus and process in accordance with the present invention.

### DESCRIPTION OF CERTAIN EMBODIMENTS

To provide an overall understanding of the invention, certain illustrative embodiments will now be described, including systems and methods for co-extruding sustained release devices, and devices fabricated according to these systems and methods. However, it will be understood that the systems and methods described herein may be usefully applied to a number of different devices, such as devices with various cross-sectional geometries or devices with two-or more concentrically aligned or non-concentrically aligned cores of different active agents. All such embodiments are intended to fall within the scope of the invention described herein.

Referring to the drawing figures, like reference numerals designate identical or corresponding elements throughout the several figures.

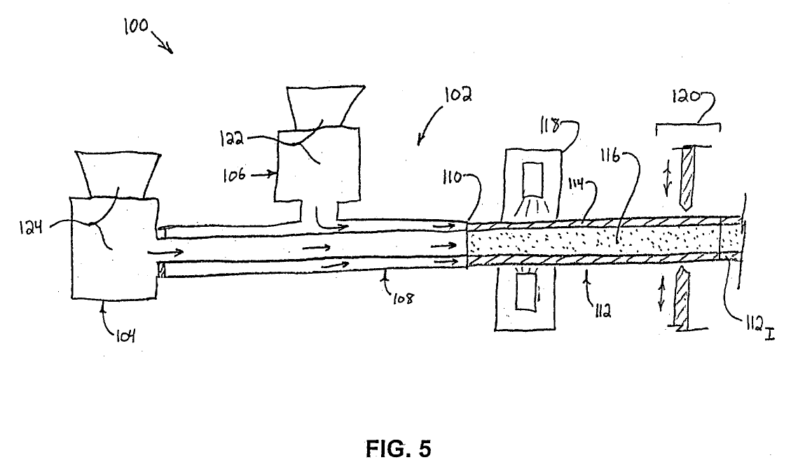

FIG. 5 illustrates an exemplary system 100 useful for performing processes in accordance with the present invention. As illustrated in FIG. 5, the system 100 may include a co-extrusion device 102 having at least a first extruder 104 and a second extruder 106, both of which are connected to a die head 108 in a manner well known to those of skill in the extrusion arts. The die head 108 has an exit port 110 out of which the co-extruded materials from the extruders 104, 106 are forced. The die head 108 may establish a cross-sectional shape of extruded matter. Many extruders are potentially useable as extruders 104, 106, including the commercially available Randcastle model RCP-0250 Microtruder (Randcastle Extrusion Systems, Cedar Grove, N.J.), and its associated heaters, controllers, and the like. See also U.S. Pat. Nos. 5,569,429, 5,518,672, and 5,486,328, for other exemplary extruders.

The extruders 104, 106 each extrude a material through the die head 108 in a known manner, forming a composite co-extruded product 112 which exits the die head at the exit 110. In a further embodiment, the extruders 104, 106 may each extrude more than one material through the die head 108 to form a composite co-extruded product 112. The system 100 may also have more than two extruders for extruding, e.g., adjacent or concentric drug matrices or additional outer layers. The product 112 includes an outer tube or skin 114 and an inner core 116. As described in greater detail herein, the outer tube 114 may be (or be the precursor to) the drug impermeable tube 112, 212, and/or 312 in the aforementioned '972 patent's devices, and the core 116 may be (or may be the precursor to) the reservoir 114, 214, and/or 314 in the '972 patent's devices.

As will be readily appreciated by those of skill in the art, extrusion processes can be highly controlled in terms of fluid pressure, flow rate, and temperature of the material being extruded. Suitable extruders may be selected for the ability to deliver the co-extruded materials at pressures and flow rates sufficient to form the product 112 at sizes of the die head which will produce a product which, when segmented, can be implanted, injected or otherwise administrable in a patient. As described in greater detail below, the materials extruded through the extruders 104, 106 also will dictate certain additional performance and operational conditions of the extruders and the extrusion process, as well as of the system 100.

The system 100 may include additional processing devices which further process the materials extruded by the extruders 104, 106, and/or the product 112. By way of example and not of limitation, the system 100 may optionally further include a curing station 118 which at least partially cures the product 112 as it passes through the station. Also further optionally, a segmenting station 120 may be provided which segments or otherwise cuts the product 112 into a series of shorter products 1121.

Materials 122, 124, suitable to form tube 114 and core 116, respectively, are numerous. In this regard, the '972 patent describes suitable materials for forming implantable drug delivery devices, which materials are included among those usable as materials 122, 124. Preferably, the materials used as materials 122, 124 are selected for their ability to be extruded through the system 100 without negatively affecting the properties for which they are specified. For example, for those materials which are to be impermeable to the drug delivered out of the drug reservoir, a material is selected which, upon being processed through an extrusion device, is or remains impermeable. Similarly, biocompatible materials are preferably chosen for the materials which will, when the drug delivery device is fully constructed, come in contact with the patient's biological tissues. Suitable materials include poly(caprolactone) (PCL), ethylene vinyl acetate polymer (EVA), poly(ethylene glycol) (PEG), poly(vinyl acetate) (PVA), poly(lactic acid) (PLA), poly(glycolic acid) (PGA), poly(lactic-co-glycolic acid) (PLGA), polyalkyl cyanoacralate, polyurethane, nylons, or copolymers thereof. In polymers including lactic acid monomers, the lactic acid may be D-, L-, or any mixture of D- and L-isomers.

The selection of the material(s) 124 which are fed into the extruder 104 to form the inner drug core 116 may raise additional concerns. As one of skill in the art readily appreciates, extrusion devices typically include one or more heaters and one or more screw drives, plungers, or other pressure-generating devices; indeed, it may be a goal of the extruder to raise the temperature, fluid pressure, or both, of the material being extruded. This can present difficulties when a pharmaceutically active drug included in the materials being processed and extruded by the extruder 104 is heated and/or exposed to elevated pressures. This difficulty can be compounded when the drug itself is to be held in a polymer matrix, and therefore a polymer material is also mixed and heated and/or pressurized with the drug in the extruder 104. The materials 124 may be selected so that the activity of the drug in the inner core 116 of the product 112 is sufficient for producing the desired effect when implanted, injected or otherwise administered in a patient. Furthermore, when the drug is admixed with a polymer for forming a matrix upon extrusion, the polymer material which forms the matrix is advantageously selected so that the drug is not destabilized by the matrix. Preferably, the matrix material is selected so that diffusion through the matrix has little or no effect on the release rate of the drug from the matrix. Also, the particle size of the drug(s) used in the matrix may have a controlling effect on dissolution of the drug(s).

The materials 122, 124, from which the product 112 is co-extruded, may be selected to be stable during the release period for the drug delivery device. The materials may optionally be selected so that, after the drug delivery device has released the drug for a predetermined amount of time, the drug delivery device erodes in situ, i.e., is bioerodible. The materials may also be selected so that, for the desired life of the delivery device, the materials are stable and do not significantly erode, and the pore size of the materials does not change.

In general, the material selection process for material 124 may proceed as follows: (1) one or more drugs are selected; (2) an extrudable material or class of materials is selected; (3) the material or class of materials is evaluated to ascertain whether it affects the release rate of the chosen drug(s) from the material or class of materials; (4) the stability and physico-chemical properties of the material or class of materials are evaluated; and (5) the material or class of materials is evaluated to ascertain whether, when formed into a matrix with the chosen drug(s), the material or class of materials prevents biological molecules (e.g., proteinaceous materials) from migrating into the matrix and affecting the release rate by, e.g., destabilizing the drug(s). Thus, there are at least two functions of the inner material: to permit co-extrusion of the core; and to inhibit, or prevent, erosion of the drug in the core. An advantage of the system is that the differences between the release rates of drug from delivery devices into different types of tissues can be minimized, thus permitting the delivery devices to be implanted, injected or otherwise administered into different types of tissues with minimal concern that drug delivery will be changed solely by the tissue type.

Material 124 may include one or multiple pharmaceutically active drugs, matrix-forming polymers, any biomaterials such as lipids (including long chain fatty acids) and waxes, anti-oxidants, and in some cases, release modifiers (e.g., water). These materials should be biocompatible and remain stable during the extrusion processes. The blend of active drugs and polymers should be extrudable under the processing conditions. The matrix-forming polymers or any biomaterials used should be able to carry a sufficient amount of active drug or drugs to produce therapeutically effective actions over the desired period of time. It is also preferred that the materials used as drug carriers have no deleterious effect on the activity of the pharmaceutical drugs.

The polymers or other biomaterials used as active drug carriers may be selected so that the release rate of drugs from the carriers are determined by the physico-chemical properties of the drugs themselves, but not by the properties of the drug carriers. The active drug carrier may also be selected to be a release modifier, or a release modifier may be added to tailor the release rate. For example, organic acid, such as citric acid and tartaric acid, may be used to facilitate the diffusion of weak basic drugs through the release medium, while the addition of amines such as triethanolamine may facilitate the diffusion of weak acidic drugs. Polymers with an acidic or basic pH value may also be used to facilitate or attenuate the release rate of active drugs. For example, poly (lactide-co-glycolide) (PLGA) may provide an acidic micro-environment in the matrix, since it has an acidic pH value after hydrolysis. For a hydrophobic drug, a hydrophilic agent may be included to increase its release rate.

Processing parameters for co-extrusion will now be discussed in greater detail.

Temperature: The processing temperature (extrusion temperature) should be below the decomposition temperatures of active drug, polymers, and release modifiers (if any). The temperature may be set at which the matrix-forming polymers are capable of accommodating a sufficient amount of active drug to achieve the desired drug loading. For example, PLGA can carry up to 55% of flucinolone acetonide (FA) when the drug-polymer blends are extruded at 100° C., but 65% at 120° C. The drug-polymer blends should display good flow properties at the processing temperature to ensure the uniformity of the final products and to achieve the desired draw ratio so the size of the final products can be well controlled.

Screw Speed: The screw speeds for the two extruders in the co-extrusion system may be set at speeds at which a predetermined amount of polymeric skin is co-extruded with the corresponding amount of drug-core materials to achieve the desired thickness of polymeric skin. For example: 10% weight of PCL (polycaprolactone) skin and 90% weight of FA/PCL drug core can be produced by operating extruder 106 at a speed nine times slower than that of extruder 104 provided that the extruders 104 and 106 have the same screw size.

A drug or other compound can be combined with a polymer by dissolving the polymer in a solvent, combining this solution with the drug or other compound, and processing this combination as necessary to provide an extrudable paste. Melt-granulation techniques, including solventless melt-granulation, with which those of skill in the art are well acquainted, may also be employed to incorporate drug and polymer into an extrudable paste.

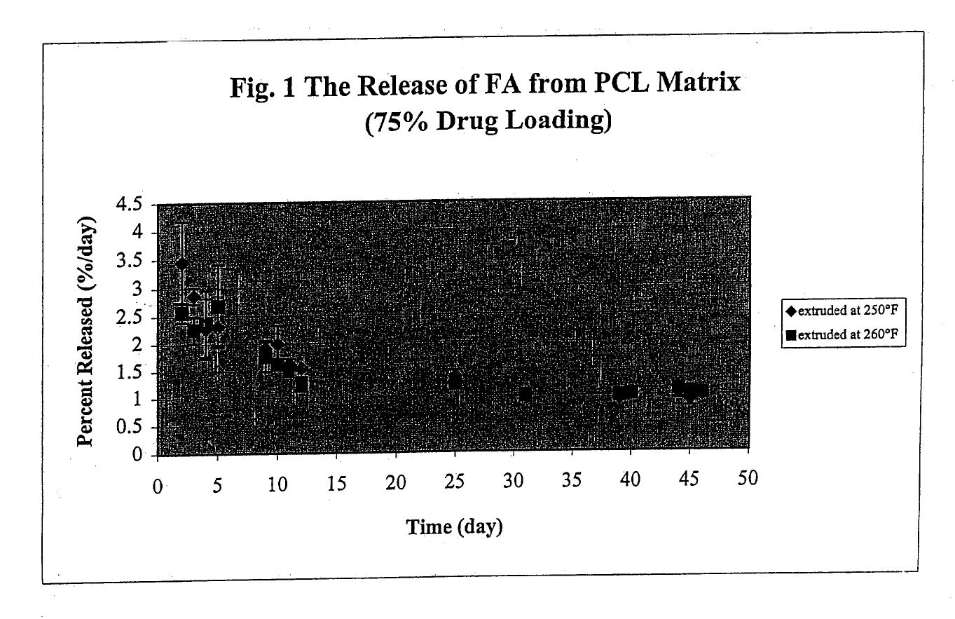

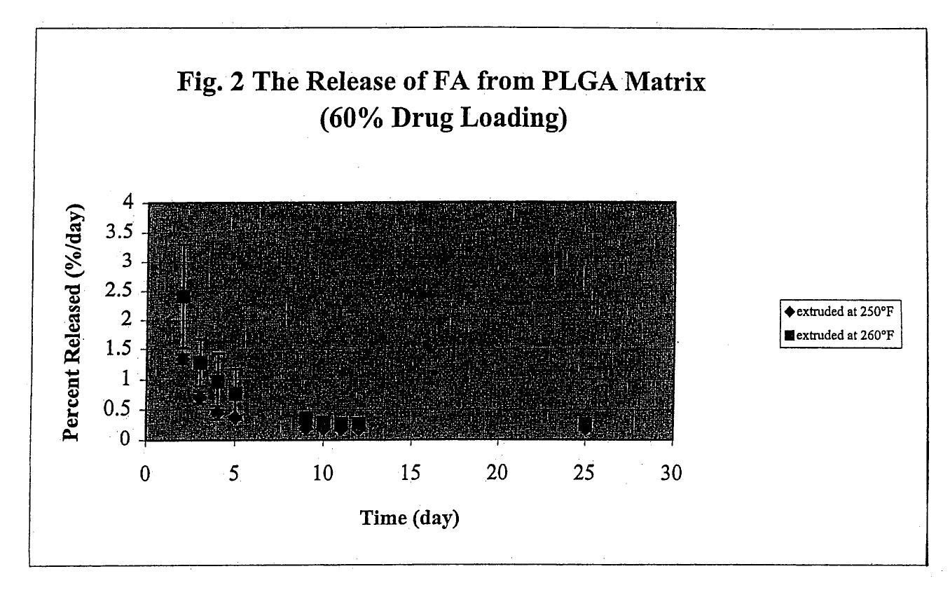

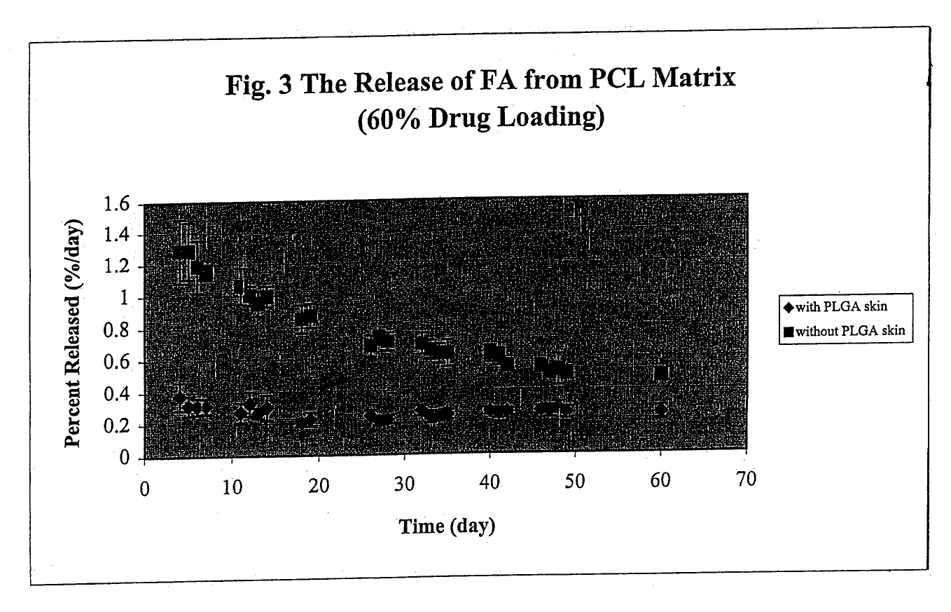

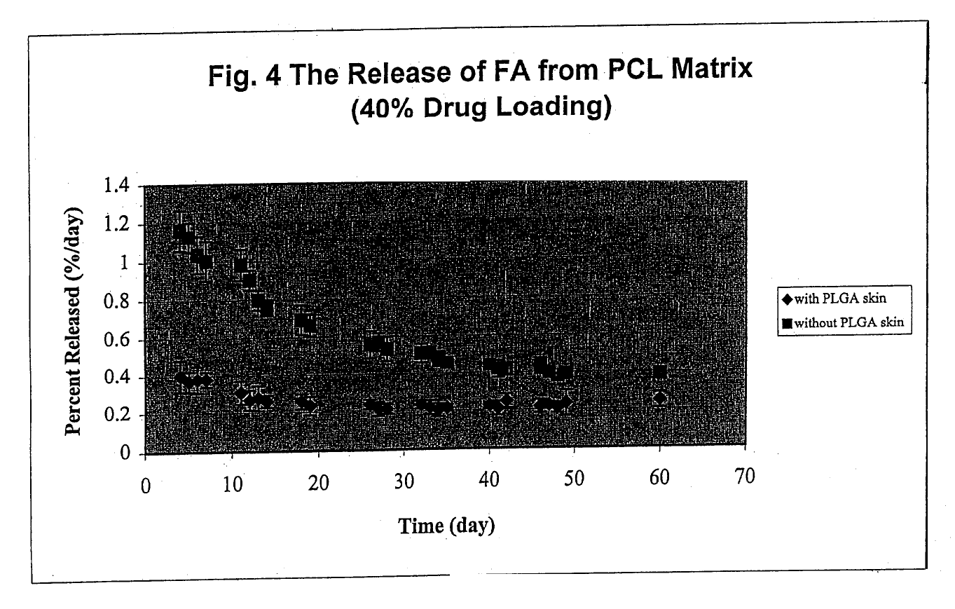

The release rate of FA from a FA/PCL (e.g., 75/25) or FA/PLGA (e.g., 60/40) core matrix with no co-extruded polymeric skin both showed a bi-phase release pattern: a burst release phase, and a slow release phase (see FIGS. 1 and 2). The burst release phase was less pronounced when FA levels (loading) in the PCL matrix were reduced from 75% to 60% or 40% (compare FIG. 1 with FIGS. 2-4). A review of the data presented in FIGS. 3 and 4 reveals that the time to reach near zero-order release for the co-extrusion preparation (drug in a polymer matrix with a PLGA skin) was much shorter than the preparation without a PLGA skin coat. Therefore, a co-extruded FA/polymer core matrix with PLGA as a skin coat can significantly minimize the burst effect, as demonstrated by FIGS. 3 and 4.

The segmented drug delivery devices may be left open on one end, leaving the drug core exposed. The material 124 which is co-extruded to form the drug core 116 of the product 112, as well as the co-extrusion heats and pressures and the curing station 118, are selected so that the matrix material of the drug core inhibits, and preferably prevents, the passage of enzymes, proteins, and other materials into the drug core which would lyse the drug before it has an opportunity to be released from the device. As the core empties, the matrix may weaken and break down. Then, the tube 114 will be exposed to degradation from both the outside and inside from water and enzymatic action. Drugs having higher solubilities are preferably linked to form low solubility conjugates; alternatively, drugs may be linked together to form molecules large enough to be retained in the matrix.

The material 122, from which the outer tube 114 is formed, may be selected to be curable by a non-heat source. As described above, it is common for drugs to be negatively affected by high temperatures. Thus, one aspect of the system relates to the selection and extrusion of a material which can be cured by methods other than heating, including, but not limited to, catalyzation, radiation and evaporation. By way of example and not of limitation, materials capable of being cured by electromagnetic (EM) radiation, e.g., in the visible or near-visible ranges, e.g., of ultraviolet or blue wavelengths, may be used, or included in, material 122. In this example, curing station 118 includes one or more sources of the EM radiation which cure the material, such as an intense light source, a tuned laser, or the like, as the product 112 advances through the station. By way of example and not of limitation, curable acrylic based adhesives may be used as material 122.

Other parameters may affect the release rate of drug from the drug core of an implantable, injectable or otherwise administrable drug delivery device, such as the pH of the core matrix. The materials 124 of the drug core may include a pH buffer or the like to adjust the pH in the matrix to further tailor the drug release rate in the finished product.

For example, organic acid, such as citric, tartaric, and succinic acid may be used to create an acidic microenvironment pH in the matrix. The constant low pH value may facilitate the diffusion of weak basic drug through the pores created upon dissolution of the drug. In the case of a weak acidic drug, an amine, such as triethanolamine, may be used to facilitate drug release rates. A polymer may also be used as a pH-dependent release modifier. For example, PLGA may provide an acidic micro-environment in the matrix as it has an acid pH value after hydrolysis.

More than one drug may be included in the material 124, and therefore in the inner core 116 of the product 112. The drugs may have the same or different release rates. As an example, 5-fluorouracil (5-FU) is highly water-soluble and it is very difficult to provide an environment where the compound can be released at a controlled rate over a sustained period. On the other hand, steroids such as triamcinolone acetonide (TA) are much more lipophilic and may provide a slower release profile. When a mixture of 5-FU and TA forms a pellet (either by compression or by co-extrusion), the pellet provides a controlled release of 5-FU over a 5-day period to give an immediate, short-term pharmaceutical effect while simultaneously providing a controlled release of TA over a much longer period. Accordingly, a mixture of 5-FU and TA, and/or prodrugs thereof, alone or with other drugs and/or polymeric ingredients, may be extruded to form inner core 116.

Codrugs or prodrugs may be used to deliver drugs in a sustained manner, and may be adapted to use in the inner core or outer skin of the drug delivery devices described above. An example of sustained-release systems using co-drugs and pro-drugs may be found in U.S. Pat. No. 6,051,576. This reference is incorporated in its entirety herein by reference.

As used herein, the term “codrug” means a first constituent moiety chemically linked to at least one other constituent moiety that is the same as, or different from, the first constituent moiety. The individual constituent moieties are reconstituted as the pharmaceutically active forms of the same moieties, or codrugs thereof, prior to conjugation. Constituent moieties may be linked together via reversible covalent bonds such as ester, amide, carbamate, carbonate, cyclic ketal, thioester, thioamide, thiocarbamate, thiocarbonate, xanthate and phosphate ester bonds, so that at the required site in the body they are cleaved to regenerate the active forms of the drug compounds.

As used herein, the term “constituent moiety” means one of two or more pharmaceutically active moieties so linked as to form a codrug according to the present invention as described herein. In some embodiments according to the present invention, two molecules of the same constituent moiety are combined to form a dimer (which may or may not have a plane of symmetry). In the context where the free, unconjugated form of the moiety is referred to, the term “constituent moiety” means a pharmaceutically active moiety, either before it is combined with another pharmaceutically active moiety to form a codrug, or after the codrug has been hydrolyzed to remove the linkage between the two or more constituent moieties. In such cases, the constituent moieties are chemically the same as the pharmaceutically active forms of the same moieties, or codrugs thereof, prior to conjugation.

The term “prodrug” is intended to encompass compounds that, under physiological conditions, are converted into the therapeutically active agents of the present invention. A common method for making a prodrug is to include selected moieties, such as esters, that are hydrolyzed under physiological conditions to convert the prodrug to an active biological moiety. In other embodiments, the prodrug is converted by an enzymatic activity of the host animal. Prodrugs are typically formed by chemical modification of a biologically active moiety. Conventional procedures for the selection and preparation of suitable prodrug derivatives are described, for example, in Design of Prodrugs, ed. H. Bundgaard, Elsevier, 1985.

In the context of referring to the codrug according to the present invention, the term “residue of a constituent moiety” means that part of a codrug that is structurally derived from a constituent moiety apart from the functional group through which the moiety is linked to another constituent moiety. For instance, where the functional group is —NH₂, and the constituent group forms an amide (—NH—CO—) bond with another constituent moiety, the residue of the constituent moiety is that part of the constituent moiety that includes the —NH— of the amide, but excluding the hydrogen (H) that is lost when the amide bond is formed. In this sense, the term “residue” as used herein is analogous to the sense of the word “residue” as used in peptide and protein chemistry to refer to a residue of an amino acid in a peptide.

Codrugs may be formed from two or more constituent moieties covalently linked together either directly or through a linking group. The covalent bonds between residues include a bonding structure such as:

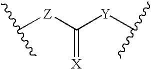

wherein Z is O, N, —CH₂—, —CH₂—O— or —CH₂—S—, Y is O, or N, and X is O or S. The rate of cleavage of the individual constituent moieties can be controlled by the type of bond, the choice of constituent moieties, and/or the physical form of the codrug. The lability of the selected bond type may be enzyme-specific. In some embodiments, the bond is selectively labile in the presence of an esterase. In other embodiments of the invention, the bond is chemically labile, e.g., to acid- or base-catalyzed hydrolysis. In some embodiments, the linking group does not include a sugar, a reduced sugar, a pyrophosphate, or a phosphate group.

The physiologically labile linkage may be any linkage that is labile under conditions approximating those found in physiologic fluids. The linkage may be a direct bond (for instance, ester, amide, carbamate, carbonate, cyclic ketal, thioester, thioamide, thiocarbamate, thiocarbonate, xanthate, phosphate ester, sulfonate, or a sulfamate linkage) or may be a linking group (for instance, a C₁-C₁₂ dialcohol, a C₁-C₁₂ hydroxyalkanoic acid, a C₁-C₁₂ hydroxyalkylamine, a C₁-C₁₂ diacid, a C₁-C₁₂ aminoacid, or a C₁-C₁₂ diamine). Especially preferred linkages are direct amide, ester, carbonate, carbamate, and sulfamate linkages, and linkages via succinic acid, salicylic acid, diglycolic acid, oxa acids, oxamethylene, and halides thereof. The linkages are labile under physiologic conditions, which generally means pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the codrug has not been solubilized in a physiologic fluid. Thus, while some codrugs may be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g., non-aqueous solvents such as acetone). Thus, the labile linkages are such that, when the codrug is dissolved in an aqueous solution, the reaction is driven to the hydrolysis products, which include the constituent moieties set forth above.

Codrugs for preparation of a drug delivery device for use with the systems described herein may be synthesized in the manner illustrated in one of the synthetic schemes below. In general, where the first and second constituent moieties are to be directly linked, the first moiety is condensed with the second moiety under conditions suitable for forming a linkage that is labile under physiologic conditions. In some cases it is necessary to block some reactive groups on one, the other, or both of the moieties. Where the constituent moieties are to be covalently linked via a linker, such as oxamethylene, succinic acid, or diglycolic acid, it is advantageous to first condense the first constituent moiety with the linker. In some cases it is advantageous to perform the reaction in a suitable solvent, such as acetonitrile, in the presence of suitable catalysts, such as carbodiimides including EDCI (1-ethyl-3-(3 -dimethylaminopropyl)-carbodiimide) and DCC (DCC: dicyclohexylcarbo-diimide), or under conditions suitable to drive off water of condensation or other reaction products (e.g., reflux or molecular sieves), or a combination of two or more thereof. After the first constituent moiety is condensed with the linker, the combined first constituent moiety and linker may then be condensed with the second constituent moiety. Again, in some cases it is advantageous to perform the reaction in a suitable solvent, such as acetonitrile, in the presence of suitable catalysts, such as carbodiimides including EDCI and DCC, or under conditions suitable to drive off water of condensation or other reaction products (e.g., reflux or molecular sieves), or a combination of two or more thereof. Where one or more active groups have been blocked, it may be advantageous to remove the blocking groups under selective conditions, however it may also be advantageous, where the hydrolysis product of the blocking group and the blocked group is physiologically benign, to leave the active groups blocked.

The person having skill in the art will recognize that, while diacids, dialcohols, amino acids, etc., are described as being suitable linkers, other linkers are contemplated as being within the present invention. For instance, while the hydrolysis product of a codrug described herein may comprise a diacid, the actual reagent used to make the linkage may be, for example, an acylhalide such as succinyl chloride. The person having skill in the art will recognize that other possible acid, alcohol, amino, sulfato, and sulfamoyl derivatives may be used as reagents to make the corresponding linkage.

Where the first and second constituent moieties are to be directly linked via a covalent bond, essentially the same process is conducted, except that in this case there is no need for a step of adding a linker. The first and second constituent moieties are merely combined under conditions suitable for forming the covalent bond. In some cases it may be desirable to block certain active groups on one, the other, or both of the constituent moieties. In some cases it may be desirable to use a suitable solvent, such as acetonitrile, a catalyst suitable to form the direct bond, such as carbodiimides including EDCI and DCC, or conditions designed to drive off water of condensation (e.g., reflux) or other reaction by-products.

The person having skill in the art will recognize that, while in most cases the first and second moieties may be directly linked in their original form, it is possible for the active groups to be derivatized to increase their reactivity. For instance, where the first moiety is an acid and the second moiety is an alcohol (i.e., has a free hydroxyl group), the first moiety may be derivatized to form the corresponding acid halide, such as an acid chloride or an acid bromide. The person having skill in the art will recognize that other possibilities exist for increasing yield, lowering production costs, improving purity, etc., of the codrug described herein by using conventionally derivatized starting materials to make the codrugs described herein.

Exemplary reaction schemes according to the present invention are illustrated in Schemes 1-4, below. These Schemes can be generalized by substituting other therapeutic agents having at least one functional group that can form a covalent bond to another therapeutic agent having a similar or different functional group, either directly or indirectly through a pharmaceutically acceptable linker. The person of skill in the art will appreciate that these schemes also may be generalized by using other appropriate linkers.

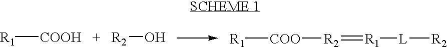

wherein L is an ester linker —COO—, and R₁ and R₂ are the residues of the first and second constituent moieties or pharmacological moieties, respectively.

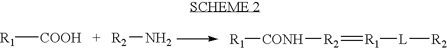

wherein L is the amide linker —CONH—, and R₁ and R₂ have the meanings given above.

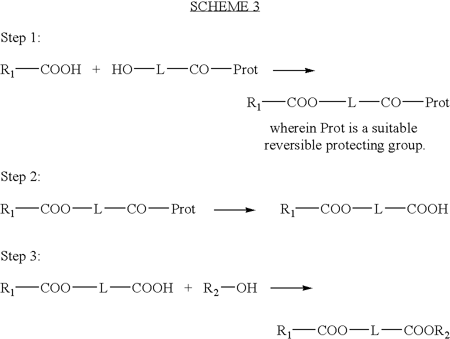

wherein R₁, L, and R₂ have the meanings set forth above.

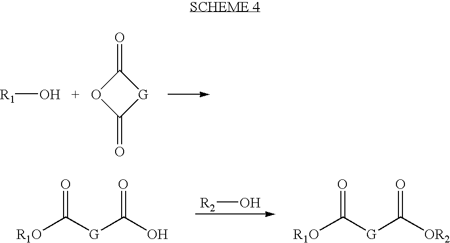

wherein R₁ and R₂ have the meanings set forth above and G is a direct bond, an C₁-C₄ alkylene, a C₂-C₄ alkenylene, a C₂-C₄ alkynylene, or a 1,2-fused ring, and G together with the anhydride group completes a cyclic anhydride. Suitable anhydrides include succinic anhydride, glutaric anhydride, maleic anhydride, diglycolic anhydride, and phthalic anhydride.

Drugs may also be included in the material 122, and therefore incorporated in the outer layer 114. This may provide biphasic release with an initial burst such that when such a system is first placed in the body, a substantial fraction of the total drug released is released from layer 114. Subsequently, more drug is released from the core 116. The drug(s) included in the outer layer 114 may be the same drug(s) as inside the core 116. Alternatively, the drugs included in the outer layer 114 may be different from the drug(s) included in the core 116. For example, the inner core 116 may include 5-FU while the outer layer 114 may include TA or loteprednol etabonate.

As noted in certain examples above, it will be appreciated that a variety of materials may be used for the outer tube or skin 114 to achieve different release rate profiles. For example, as discussed in the aforementioned '972 patent, an outer layer (such as the skin 114) may be surrounded by a permeable or impermeable outer layer (element numbers 110, 210, and 310 in the '972 patent), or may itself be formed of a permeable or semi-permeable material. Accordingly, co-extruded devices may be provided with one or more outer layers using techniques and materials fully described in the '972 patent. Through these permeable or semi-permeable materials, active agents in the core may be released at various rates. In addition, even materials considered to be impermeable may permit release of drugs or other active agents in the core 116 under certain circumstances. Thus, permeability of the outer tube 114 may contribute to the release rate of an active agent over time, and may be used as a parameter to control the release rate over time for a deployed device.

Further, a continuous extrusion may be segmented into devices having, for example, an impermeable outer tube 114 surrounding a core, with each segment further coated by a semi-permeable or permeable layer to control a release rate through the exposed ends thereof. Similarly, the outer tube 114, or one or more layers thereof, or a layer surrounding the device, may be bioerodible at a known rate, so that core material is exposed after a certain period of time along some or all of the length of the tube, or at one or both ends thereof.

Thus, it will be appreciated that, using various materials for the outer tube 114 and one or more additional layers surrounding a co-extruded device, the delivery rate for the deployed device may be controlled to achieve a variety of release rate profiles.

Extrusion, and more particularly co-extrusion, of the product 112 permits very close tolerances of the dimensions of the product. It has been found that a significant factor affecting the release rate of drug from a device formed from the product 112 is the internal diameter (ID) of the outer tube 114, which relates to the (at least initial) total surface area available for drug diffusion. Thus, by maintaining close tolerances of tube 114's ID, the variation in release rates from the drug cores of batches of devices can be minimized.

### EXAMPLE

A co-extrusion line consisting of two Randcastle microtruders, a concentric co-extrusion die, and a conveyer is used to manufacture an injectable delivery device for FA. Micronized powder of FA is granulated with the following matrix forming material: PCL or poly(vinyl acetate) (PVAC) at a drug loading level of 40% or 60%. The resulting mixture is co-extruded with or without PLGA or polyethylene-co-vinyl acetate (EVA) as an outer layer coating to form a composite tube-shape product. In-vitro release studies were carried out using pH 7.4 phosphate buffer to evaluate the release characteristics of FA from different delivery devices.

FA granules used to form the drug reservoir were prepared by mixing 100 g of FA powder with 375 g and 167 g of 40% PCL solution to prepare 40% and 60% drug loading formulations, respectively. After oven-drying at 55° C. for 2 hours, the granules were ground to a size 20 mesh manually or using a cryogenic mill. The resulting drug/polymer mixture was used as material 124 and was co-extruded with PLGA as material 122 using two Randcastle Model RCP-0250 microextruders to form a composite co-extruded, tube-shaped product 112.

The diameter of the delivery device can be controlled by varying the processing parameters, such as the conveyor speed and the die diameter. All the preparations were capable of providing long-term sustained release of FA. The release of FA from the PCL matrix without the outer layer of polymeric coat was much faster than that with PLGA skin. It showed a bi-phase release pattern: a burst release phase followed by a slow release phase. On the other hand, the preparation with the PLGA coat gave a linear release of FA for at least five months regardless of the drug level. PLGA coating appeared to be able to minimize the burst effect significantly. It also was observed that the release rate of FA was proportional to the drug loading level in the matrix. Compared to PLGA, EVA largely retarded the release of FA. In addition to variations in release rate, it will be appreciated that different polymers may possess different physical properties for extrusion.

Co-extrusion may be used to manufacture implantable, injectable or otherwise administrable drug delivery devices. The release of drugs, such as steroids, from such devices can be attenuated by using a different combination of inner matrix-forming materials and outer polymeric materials. This makes these devices suitable for a variety of applications where controlled and sustained release of drugs, including steroids, is desired.

It is to be understood that the term “drug” as it is used in the present application is intended to encompass all agents which are designed to provide a local or systemic physiological or pharmacological effect when administered to mammals, including prodrugs thereof.

While the invention has been described in detail with reference to preferred embodiments thereof, it will be apparent to one skilled in the art that various changes can be made, and equivalents employed, without departing from the scope of the invention. Each of the aforementioned published documents is incorporated by reference herein in its entirety.

1. A method of making a drug delivery device comprising co-extruding an inner drug-containing core and at least one outer polymeric skin that at least partially surrounds the core.

2. The method of claim 1, wherein the device is at least one of insertable, injectable, or implantable.

3. The method of claim 1, wherein the inner drug-containing core comprises a mixture of at least one drug and at least one polymer.

4. The method of claim 3, wherein the polymer of the inner drug-containing core is bioerodible.

5. The method of claim 3, wherein the at least one drug and the at least one polymer are admixed in powder form.

6. The method of claim 1, wherein the device includes at least one of a codrug or a prodrug.

7. The method of claim 1, wherein the inner drug core comprises a steroid.

8. The method of claim 7, wherein the steroid includes at least one of flucinolone acetonide (FA), loteprednol etabonate, or triamcinolone acetonide (TA).

9. The method of claim 1, wherein at least one of the inner drug core or the at least one outer polymeric skin comprises an anti-metabolite.

10. The method of claim 9, wherein the anti-metabolite comprises 5-flurouracil (5-FU).

11. The method of claim 1, wherein the outer polymeric skin is one of impermeable, semi-permeable, or permeable to a drug disposed within the inner drug-containing core.

12. The method of claim 1, wherein the outer polymeric skin comprises at least one of polycaprolactone (PCL), an ethylene/vinyl acetate copolymer (EVA), polyalkyl cyanoacralate, polyurethane, a nylon, or poly(dl-lactide-co-glycolide) (PLGA).

13. The method of claim 1, wherein the inner drug-containing core comprises FA in admixture with poly(vinyl acetate) (PVAC), PCL, PEG or PLGA.

14. The method of claim 1, wherein the outer polymeric skin is bioerodible.

15. The method of claim 14, wherein the inner drug-containing core comprises a bioerodible polymer.

16. The method of claim 1, wherein the outer polymeric skin is radiation curable and the method further comprises applying radiation to the co-extruded drug delivery device.

17. The method of claim 1, wherein the outer polymeric skin comprises at least one drug.

18. The method according to claim 17, wherein the at least one drug comprises TA.

19. The method of claim 18, wherein the inner drug-containing core comprises 5-FU.

20. The method of claim 1, wherein the inner drug-containing core comprises 5-FU.

21. A method of making a drug delivery device comprising:
(a) forwarding a polymeric material to a first extrusion device;
(b) forwarding a drug to a second extrusion device;
(c) co-extruding a mass including the polymeric material and the drug; and
(d) forming the mass into at least one co-extruded drug delivery device which comprises a core including the drug and an outer layer including the polymeric material.

22. The method of claim 21, wherein the drug forwarded to the second extrusion device is in admixture with at least one polymer.

23. The method of claim 22, wherein the drug and the at least one polymer are admixed in powder form.

24. The method of claim 21, further comprising forwarding more than one drug to the second extrusion device.

25. The method of claim 21 wherein the polymeric material is one of impermeable, semi-permeable, or permeable to the drug.

26. The method of claim 21, wherein the polymeric material is bioerodible.

27. The method of claim 22, wherein the admixture with at least one polymer is bioerodible.

28. The method of claim 27, wherein the polymeric material is bioerodible.

29. The method of claim 21, wherein the polymeric material is radiation curable and the method further comprises applying radiation to the co-extruded drug delivery device.

30. The method of claim 21, wherein the co-extruded drug delivery device is in a tubular form.

31. The method of claim 21, further comprising segmenting the tubular form into a plurality of shorter products.

32. The method of claim 31, further comprising coating the plurality of shorter products with one or more layers including at least one of a layer that is permeable to the drug, a layer that is semi-permeable to the drug, and a layer that is bioerodible.

33. The method of claim 21, wherein the polymeric material includes at least one of PCL, PLGA or an EVA.

34. The method of claim 21, wherein the drug includes a steroid.

35. The method of claim 34, wherein the steroid includes at least one of FA or TA.

36. The method of claim 21, wherein the drug includes an anti-metabolite.

37. The method of claim 34, wherein the anti-metabolite is 5-FU.

38. The method of claim 37, wherein the polymeric material includes TA.

39. The method of claim 21, wherein the polymeric material includes TA.

40. The method of claim 21, wherein the drug is FA in admixture with at least one of PCL, PLGA or PVAC.

41. The method of claim 21, wherein the polymeric material includes at least one of PCL, PLGA or an EVA and the drug includes FA in admixture with at least one of PCL, PLGA or PVAC.

42. The method of claim 21, wherein the polymeric material includes at least one drug.

43. A device for fabricating an implantable drug delivery device comprising:
(a) a first extruder for extruding a core, wherein the core includes at least one drug; and
(b) a second extruder for extruding a skin, wherein the skin is disposed about the core to form a co-extruded material, and wherein the skin has at least one of a permeability or an erodibility selected to control the release rate of the drug in a device formed from a segment of the co-extruded material.

44. The device of claim 43, further comprising a segmenting station that separates the co-extruded material into a plurality of segments.

45. The device of claim 43, further comprising a curing station that at least partially cures the co-extruded material.
